(19) United States
(12) Patent Application Publication
(10) Pub. No.: US 2021/0290050 A1
(43) Pub. Date: Sep. 23, 2021

(54) OCULAR FUNDUS IMAGE PROCESSING APPARATUS

(71) Applicant: NIDEK CO., LTD., Gamagori (JP)

(72) Inventors: Ryosuke SHIBA, Gamagori (JP); Yoshiki Kumagai, Gamagori (JP); Masayuki Yoshino, Gamagori (JP)

(73) Assignee: NIDEK CO., LTD., Gamagori (JP)

(21) Appl. No.: 17/132,190

(22) Filed: Dec. 23, 2020

(30) Foreign Application Priority Data
Dec. 26, 2019 (JP) 2019-237244

(51) Int. Cl. A61B 3/00; A61B 3/12

(52) U.S. Cl. CPC A61B 3/0025; A61B 3/0008; A61B 3/12

(57) ABSTRACT

An ocular fundus image processing apparatus acquires a fundus image of an subject eye which is captured by a fundus capturing apparatus, acquires a correction image including artifact caused by illumination light without including a fundus of the subject eye as a capturing target, executes conversion processing of the correction image a plurality of times while changing processing content to acquire a plurality of conversion images, and acquires a difference image, as high-quality images, in which an influence of the artifact is suppressed to be less than or equal to a criterion, among a plurality of difference images acquired by taking a difference between each of the plurality of conversion images and the fundus image.

### CROSS-REFERENCE TO RELATED APPLICATION

This application claims priority from Japanese Patent Application No. 2019-237244 filed on Dec. 26, 2019, the entire subject-matter of which is incorporated herein by reference.

### TECHNICAL FIELD

The present disclosure relates to an ocular fundus image processing apparatus that processes a fundus image of a subject eye which is captured by a fundus capturing apparatus.

### BACKGROUND ART

Various fundus capturing apparatuses that capture a fundus image of a subject eye are known. For example, JP-S61-48940-B discloses an apparatus that scans a slit-shaped illumination light on fundus and sequentially projects an image of an illuminated fundus region onto a two-dimensional imaging surface according to scan, to obtain a front image of the fundus. Further, an apparatus (for example, a scanning laser ophthalmoscope (SLO) or the like) that scans a spot-shaped illumination light to capture a front image of a fundus is also known. Further, an apparatus (for example, a fundus camera) that simultaneously irradiates a two-dimensional region of a fundus with illumination light to capture a front image of a fundus is also known.

The fundus image captured by the fundus capturing apparatus may include various artifacts (for example, artifacts occurring when illumination light is reflected by an objective lens or the like and is incident on a light receiving element, or the like). In order to remove the artifacts included in the fundus image, there is a method of acquiring a correction image obtained by capturing only the artifacts by using the same fundus capturing apparatus and acquiring a difference image, as a high-quality image, between the fundus image and the correction image. In this method, a weight when converting the correction image is previously determined such that a correlation between the difference image and the correction image is reduced, and the difference image between the correction image converted by the weight and the fundus image is acquired.

There are many cases in which various types of processing (for example, at least one processing such as gain adjustment and gamma correction) are executed in an imaging process of generating image data based on a light receiving signal output by a light receiving element of the fundus capturing apparatus. However, in this method, information of the imaging process is not taken into consideration when converting the correction image, and thus, it may be difficult to appropriately suppress an influence of the artifacts. If the information of the imaging process is known, conversion of the correction image based on the information of the imaging process can also be considered, but there are also many cases where the information of the imaging process is hard to acquire. Further, in the above-described method, when the correction image includes an element other than the artifacts, the artifacts of the fundus image is not appropriately removed. As described above, the above-described method is hard to appropriately suppress the influence of the artifacts of the fundus image.

### SUMMARY OF INVENTION

An object of the present disclosure is to provide an ocular fundus image processing apparatus capable of appropriately suppressing an influence of artifact of a fundus image.

An ocular fundus image processing apparatus according to the present disclosure is

an ocular fundus image processing apparatus that processes a fundus image captured by a fundus capturing apparatus,

in which the fundus capturing apparatus includes

a light source that emits illumination light,

an objective lens that irradiates a downstream side of an optical path with the illumination light, and

a light receiving element that receives imaging light incident from the objective lens, and

the ocular fundus image processing apparatus comprising a controller configured to execute:

fundus image acquisition processing of acquiring a fundus image, which is an image of a fundus of a subject eye, captured by the fundus capturing apparatus,

correction image acquisition processing of acquiring a correction image, which is an image including artifact caused by the illumination light, captured by the fundus capturing apparatus without including the fundus of a subject eye as an imaging target,

conversion image acquisition processing of executing conversion processing of converting a pixel value of the correction image a plurality of times while changing processing content to acquire a plurality of conversion images, and

high-quality image acquisition processing of acquiring a difference image, as a high-quality image, in which an influence of the artifact is suppressed to be less than or equal to a criterion, among a plurality of difference images acquired by taking a difference between each of the plurality of conversion images and the fundus image.

### BRIEF DESCRIPTION OF DRAWINGS

FIG. 1 is a side view illustrating an external configuration of a fundus capturing apparatus 1 of the present embodiment.

FIG. 2 is a diagram illustrating an optical system housed in a capturing unit 3 of the present embodiment.

FIG. 3 is a block diagram illustrating a control system of the fundus capturing apparatus 1 of the present embodiment.

FIG. 4 is a diagram illustrating an example of a diopter correction state in a case where a subject eye E is an emmetropic eye.

FIG. 5 is a diagram illustrating an example of a diopter correction state in a case where the subject eye E is a myopic eye.

FIGS. 6A and 6B are flowcharts of fundus image processing executed by a fundus capturing apparatus (ocular fundus image processing apparatus) 1, which is evaluated by combining these drawings.

FIG. 7 is a diagram illustrating a fundus image 60, a correction image 70, a conversion image 80, and a difference image 90, processed by the fundus capturing apparatus (ocular fundus image processing apparatus) 1.

FIG. 8 is a modification example of the flowchart of the fundus image processing executed by the fundus capturing apparatus (ocular fundus image processing apparatus) 1.

### DESCRIPTION OF EMBODIMENTS

### Overview

An ocular fundus image processing apparatus according to the present disclosure processes a fundus image captured by a fundus capturing apparatus. The fundus capturing apparatus includes a light source, an objective lens, and a light receiving element. The light source emits illumination light. The objective lens irradiates a downstream side of an optical path with the illumination light. The light receiving element receives imaging light incident from the objective lens. A controller of the ocular fundus image processing apparatus executes fundus image acquisition processing, correction image acquisition processing, conversion image acquisition processing, and high-quality image acquisition processing. In the fundus image acquisition processing, the controller acquires the fundus image of a subject eye which is captured by the fundus capturing apparatus. In the correction image acquisition processing, the controller acquires a correction image which is an image captured by the fundus capturing apparatus and is an image including artifact caused by the illumination light without including a fundus of the subject eye as a capturing target. In the conversion image acquisition processing, the controller executes conversion processing of converting a pixel value of the correction image a plurality of times while changing processing content to acquire a plurality of conversion images. In the high-quality image acquisition processing, among a plurality of difference images acquired by taking a difference between each of the plurality of conversion images and the fundus image, the controller acquires a difference image, as a high-quality image, in which an influence of the artifact is suppressed to be less than or equal to a criterion.

The correction image acquired in the correction image acquisition processing according to the present disclosure includes only the artifact (for example, artifact occurring when illumination light is reflected by the objective lens or the like and is incident on the light receiving element, or the like). The controller of the ocular fundus image processing apparatus execute the conversion processing a plurality of times for the correction image while changing processing content to acquire a plurality of conversion images. The controller searches for the difference image in which an influence of the artifact is suppressed to be less than or equal to the criterion, among the plurality of difference images acquired by taking a difference between the fundus image and each of the plurality of conversion images, and acquires the searched difference image as a high-quality image. Thus, even in a case where information of imaging process is unknown, the artifact of the fundus image is removed by the conversion image obtained by appropriately executing the conversion processing based on the information of the imaging process. Thus, an influence of the artifact of the fundus image is appropriately removed.

Various fundus images can be adopted in the fundus image processed by the ocular fundus image processing apparatus. For example, the fundus image may be a two-dimensional front image obtained by capturing a fundus in a line-of-sight direction of the subject eye. In this case, the fundus capturing apparatus may be a fundus camera that captures a two-dimensional front image of a fundus by simultaneously irradiating two-dimensional regions of the fundus with illumination light. Further, the fundus capturing apparatus may be a slit scan type capturing apparatus that captures a two-dimensional front image of a fundus by scanning slit-shaped illumination light in a direction crossing an extension direction of a slit. Further, the fundus capturing apparatus may be a capturing apparatus (for example, scanning laser ophthalmoscope (SLO) or the like) that captures a two-dimensional front image of a fundus by irradiating the fundus with spot-shaped illumination light and scanning the spot-shaped illumination light two-dimensionally.

The correction image may be an image captured when the fundus capturing apparatus emits the illumination light in a state where light from the downstream side of the objective lens toward the objective lens in the optical path of the illumination light is blocked. As an example, the correction image may be captured in a state where the illumination light of the downstream side rather than the objective lens, in the fundus capturing apparatus, is blocked by a cover or the like. Further, the correction image may be captured by capturing a blackout curtain or the like by using the fundus capturing apparatus. In this case, the correction image includes only artifact without including a capturing target such as the fundus.

In the high-quality image acquisition processing, the controller may acquire the plurality of difference images by taking a difference between each of the plurality of conversion images and the fundus image. The controller may search for the difference image, among the plurality of difference images, of which a correlation with a conversion image or a correction image is less than or equal to a criterion, and may acquire the searched difference image as the high-quality image. In this case, the high-quality image in which the influence of the artifact is suppressed is appropriately searched for by the correlation between the difference image and the conversion image or the correction image.

However, a specific method for acquiring the high-quality image can also be changed. For example, the controller may search for the conversion image, among the plurality of conversion images, of which the correlation with the fundus image is greater than or equal to a criterion, and may acquire the difference image between the searched conversion image and the fundus image as the high-quality image. Even in this case, the high-quality image in which the influence of the artifact is suppressed is appropriately searched for by the correlation between the conversion image and the fundus image.

The fundus capturing apparatus may further include a diopter correction portion that performs diopter correction according to the subject eye in conjunction with or separately from the illumination light and the imaging light. The correction image acquired in the correction image acquisition processing may be an image captured in a state where diopter correction is made with a diopter correction amount in which a difference from the diopter correction amount by the diopter correction portion when capturing the fundus image is less than or equal to a threshold. In this case, since the fundus image and the correction image are captured by an approximate diopter correction amount (focus adjustment amount), the artifact included in the fundus image and the artifact included in the correction image can be easily approximated. Thus, the influence of the artifact of the fundus image is removed more appropriately.

Among image capturing conditions when capturing the fundus image and the correction image, the image capturing conditions other than the diopter correction amount may be the same. For example, among the image capturing conditions when capturing the fundus image and the correction image, at least one of amount of the illumination light and an image capturing range for capturing an image may be the same. In this case, the influence of the artifact of the fundus image is removed more appropriately.

If the subject eye is constantly an emmetropic eye, an optical system can be designed such that a position (that is, a position of an intermediate image surface of the fundus) conjugate with the fundus with respect to the objective lens is always on an upstream side of the illumination light more than the objective lens. In this case, since a light collection position of the illumination light is on the upstream side of the illumination light with respect to the objective lens, the amount of the illumination light reflected by the objective lens and guided to the light receiving element is hard to increase. Thus, occurrence of the artifact caused by the objective lens is appropriately suppressed. However, in order to capture a high-quality front image of the fundus, it is necessary to perform diopter correction according to the subject eye by the diopter correction portion. By performing the diopter correction, for example, it is possible to suppress an error in an illumination range and to make a uniform distribution of the amount of the illumination light in the fundus. Meanwhile, when the diopter correction is performed, the fundus conjugate position moves along an optical axis. In a case where the subject eye is a myopic eye, when the diopter correction is performed, the fundus conjugate position (that is, light collection position of the illumination light) approaches the objective lens along the optical axis. As a result, the influence of the artifact in the fundus image becomes stronger. The more the refractive index of the subject eye is a value on a minus diopter side (that is, the stronger the degree of myopia of a subject eye), the stronger the influence of the artifact caused by the objective lens.

Thus, the controller may execute the correction image acquisition processing, the conversion image acquisition processing, and the high-quality image acquisition processing only in a case where the diopter correction amount (a refractive index of the subject eye which is corrected by the diopter correction portion) when the fundus image is captured is a value on a minus diopter side of a threshold. In this case, processing for suppressing the influence of the artifact is executed for the fundus image in which the refractive index of the subject eye is the value on the minus diopter side of the threshold and the influence of the artifact is likely to be strong. Meanwhile, in a case where the diopter correction amount is not the value on the minus diopter side of the threshold and the artifact is hard to be included in the fundus image, various types of processing using the correction image are omitted. Thus, amount of processing of the ocular fundus image processing apparatus is appropriately reduced.

In a case where there is a saturation (overexposure) in a portion (for example, a bright spot or the like) of the artifact in the fundus image, even when the high-quality image acquisition processing is executed, it is difficult to restore an image of a fundus tissue by removing the artifact in the fundus image. Thus, in a case where the diopter correction amount is a value on the minus diopter side of the threshold, the fundus capturing apparatus may limit at least one of the amount of the illumination light, gain, and exposure time compare with a case where the diopter correction amount is a value on a plus diopter side of the threshold. In this case, since the saturation (overexposure) of the fundus image is suppressed, the image of the fundus tissue is restored appropriately by executing the high-quality image acquisition processing.

A plurality of correction images captured by the fundus capturing apparatus while changing the diopter correction amount by using the diopter correction portion may be previously stored in a storage apparatus. In the correction image acquisition processing, the correction image which is captured in a state where the diopter correction is made with a diopter correction amount similar to the amount at the time capturing the fundus image may be acquired, among the plurality of correction images stored in the storage apparatus. In this case, since there is no need to capture the correction image when capturing the fundus image, a work amount, image capturing time, and the like are appropriately reduced when capturing the fundus image.

However, it is also possible to change a method of acquiring the correction image. For example, the controller of the ocular fundus image processing apparatus may output an execution instruction for capturing the correction image to the fundus capturing apparatus when executing the high-quality image acquisition processing. That is, the fundus capturing apparatus may capture the correction image when capturing the fundus image. In this case, since an image capturing condition at the time of capturing the fundus image and an image capturing condition at the time of capturing the correction image are more easily approximated, the artifact included in the fundus image and the artifact included in the correction image are more easily approximated. Thus, the influence of the artifact included in the fundus image is suppressed more appropriately. 100291 In the high-quality image acquisition processing, in a case where there is no difference image in which the influence of the artifact is less than or equal to a criterion, the controller may execute re-capturing processing of outputting a re-capturing instruction of at least one of the fundus image and the correction image or may execute warning processing of warning a user that image quality of the fundus image is low. In a case where the influence of the artifact is not less than or equal to the criterion, there is a possibility that at least one of the fundus image and the correction image is not been captured appropriately. Thus, by re-capturing at least one of the fundus image and the correction image, the high-quality image can be appropriately acquired in the high-quality image acquisition processing to be executed thereafter. Further, by executing the warning processing, a user can easily grasp that the image quality of the acquired fundus image is low.

In a case where the correction image is stored in the storage apparatus and the correction image is re-captured, the re-captured correction image may be newly stored in the storage apparatus. In this case, since an appropriate correction image is newly stored according to a state (for example, the amount of light from the light source, or the like) of the fundus capturing apparatus at that time, the influence of the artifact of the fundus image is more appropriately removed.

Further, a method of determining whether or not there is the difference image in which the influence of the artifact is less than or equal to a criterion can be appropriately selected. For example, the controller may determine whether or not there is the difference image of which a correlation with the conversion image or the correction image is less than or equal to a criterion. Further, the controller may determine whether or not there is the conversion image of which the correlation with the fundus image is greater than or equal to a criterion, thereby determining whether or not there is the difference image in which the influence of the artifact is less than or equal to the criterion.

In a case where a luminance value of the artifact in the fundus image acquired in the fundus image acquisition processing greater than or equal to a threshold, the controller may output the re-capturing instruction of the fundus image. As described above, in a case where there is the saturation (overexposure) in the portion (for example, a bright spot or the like) of the artifact in the fundus image, even when the high-quality image acquisition processing is executed, it is difficult to restore an image of the fundus tissue by removing the artifact in the fundus image. Thus, by re-capturing the fundus image in a case where the luminance value of the artifact in the fundus image is greater than or equal to the threshold, a high-quality fundus image can be appropriately acquired.

The conversion processing executed in the conversion image acquisition processing may include at least one of gamma correction, brightness correction, contrast enhancement, histogram extension, and histogram equalization. In this case, the correction image is appropriately converted into the conversion image such that the influence of the artifact in the difference image is reduced.

In the conversion processing, different processing may be executed for each channel (for example, RGB, CMYK, HSV, or the like) of the correction image. In this case, the correction image is converted into the conversion image such that the influence of the artifact in the difference image is reduced. Further, in the conversion processing, the same processing may be executed for each channel of the correction image.

The controller may execute the conversion processing of the conversion image acquisition processing and processing of taking a difference between the fundus image and the conversion image in the high-quality image acquisition processing, for a partial region which includes the artifact, in an entire image region of the fundus image and the correction image. In this case, since the conversion processing and a difference processing of a region where the artifact does not exist are omitted, the amount of processing is appropriately reduced. However, the controller can also execute the conversion processing or the like for the entire image region.

The controller of the ocular fundus image processing apparatus may convert the correction image into the conversion image, based on the information of the imaging process of generating data of the fundus image based on the light receiving signal output by the light receiving element of the fundus capturing apparatus. In this case, since the artifact included in the fundus image and the artifact included in the conversion image are easily approximated, the influence of the artifact in the fundus image is appropriately suppressed.

In this case, the ocular fundus image processing apparatus can also be described as follows.

An ocular fundus image processing apparatus processes a fundus image captured by a fundus image capturing apparatus,

in which the fundus image capturing apparatus includes

a light source that emits illumination light,

an objective lens that irradiates a downstream side of an optical path with the illumination light, and

a light receiving element that receives illumination light incident from the objective lens, and

a controller of the ocular fundus image processing apparatus executes

fundus image acquisition processing of acquiring a fundus image, which is an image of a fundus of a subject eye, captured by the fundus image capturing apparatus,

correction image acquisition processing of acquiring a correction image which is an image including artifact caused by the illumination light, captured by the fundus capturing apparatus without including a fundus of a subject eye as a capturing target,

imaging information acquisition processing of acquiring information of an imaging process when data of the fundus image is generated based on a light receiving signal output by the light receiving element,

conversion image acquisition processing of executing conversion processing of converting a pixel value of the correction image based on the information of the imaging process to acquire a conversion image, and

high-quality image acquisition processing of acquiring a difference image of the fundus image and the conversion image as a high-quality image in which an influence of artifact of the fundus image is suppressed.

### Embodiment

Hereinafter, one of typical embodiments according to the present disclosure will be described. A fundus capturing apparatus 1 of the present embodiment captures a fundus image and a correction image of a subject eye and processes the fundus image based on the correction image. That is, the fundus capturing apparatus of the present embodiment also functions as an ocular fundus image processing apparatus that processes the fundus image. However, a configuration of the ocular fundus image processing apparatus can also be changed. For example, any one of a personal computer (hereinafter, referred to as “PC”), a server, a mobile terminal, a smartphone, and the like may acquire the fundus image and the correction image captured by the fundus capturing apparatus 1 to process the fundus image. That is, an information processing apparatus different from the fundus capturing apparatus may function as the ocular fundus image processing apparatus.

Further, as an example, the fundus capturing apparatus 1 of the present embodiment irradiates a fundus of the subject eye with illumination light in a slit shape and scans the illumination light in a direction crossing an extension direction of a slit. The fundus capturing apparatus 1 captures a two-dimensional front image of the fundus by receiving fundus reflection light of the illumination light. That is, the fundus capturing apparatus 1 of the present embodiment captures the fundus image by using a slit scan method. However, as described above, a method of capturing the fundus image is not limited to the slit scan method.

### Appearance of Apparatus

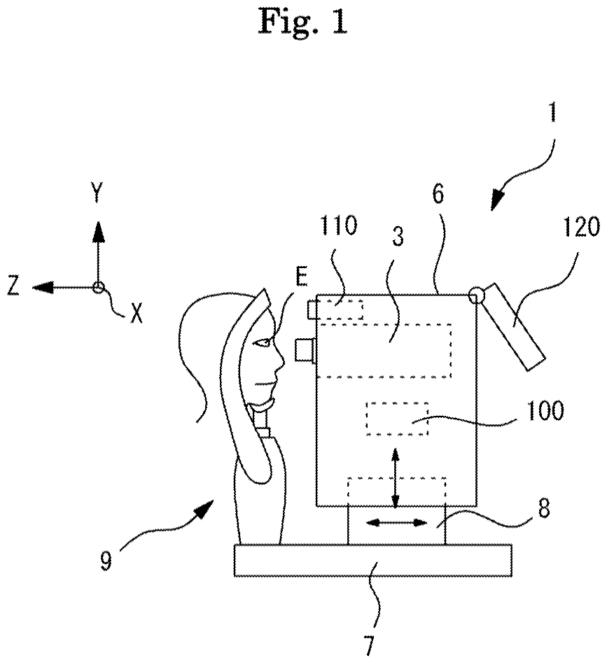

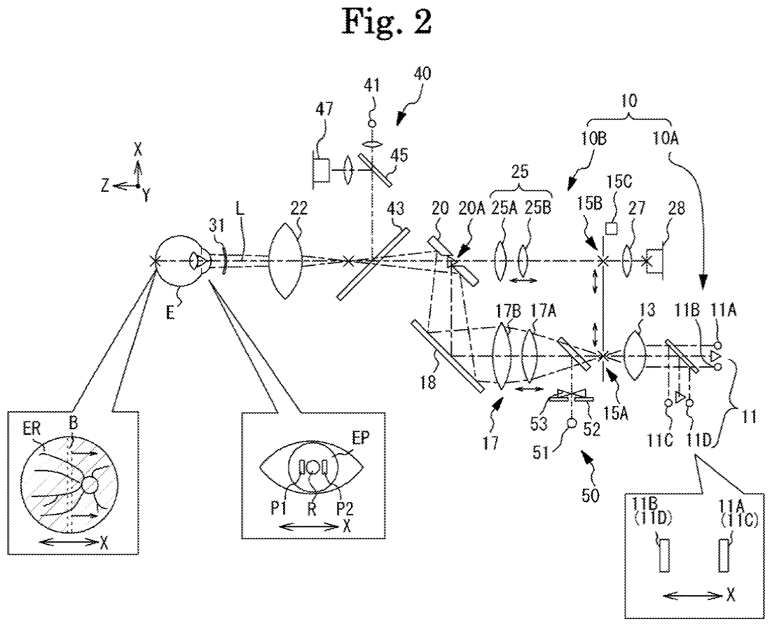

An external configuration of the fundus capturing apparatus 1 will be described with reference to FIG. 1. The fundus capturing apparatus 1 includes a capturing unit 3. The capturing unit 3 includes an optical system illustrated in FIG. 2. The fundus capturing apparatus 1 includes a housing 6, a base 7, a drive portion 8, a face support unit 9, and a face capture camera 110, and adjusts a positional relation between a subject eye E and the capturing unit 3 by using the above-described portions.

The drive portion 8 can move the capturing unit 3 in a left-right direction (X direction), a vertical direction (Y direction), and a front-rear direction (Z direction, in other words, working distance direction) with respect to the base 7. That is, the drive portion 8 moves relative positions of the capturing unit 3 and the subject eye E in a three-dimensional direction. The drive portion 8 includes an actuator for moving the capturing unit 3 in each predetermined movable direction. The drive portion 8 drives based on a control signal from a controller 100. The face support unit 9 supports a face of an examinee. The face support unit 9 is fixed to the base 7.

The face capture camera 110 is fixed to the housing 6 such that a positional relation on the capturing unit 3 is constant. The face capture camera 110 captures the face of the examinee. The controller 100 specifies a position of the subject eye E from a captured face image and performs a drive control of the drive portion 8, thereby aligning the capturing unit 3 with respect to the specified position of the subject eye E. Further, the fundus capturing apparatus 1 includes a monitor 120. The monitor (display portion) 120 displays various images (for example, fundus observation image, fundus capturing image (fundus image), front eye portion observation image, and the like).

### Optical System

An optical system of the fundus capturing apparatus 1 will be described with reference to FIG. 2. The fundus capturing apparatus 1 includes an image capturing optical system (fundus capturing optical system) 10, and a front eye portion observation optical system 40. The image capturing optical system 10 and the front eye portion observation optical system 40 are provided in the capturing unit 3. In FIG. 2, a position that conjugates with a pupil of a subject eye is denoted by “Δ” on an image capturing optical axis, and a fundus conjugate position is denoted by “x” on the image capturing optical axis.

The image capturing optical system 10 includes an irradiation optical system 10A and a light receiving optical system 10B. The irradiation optical system 10A of the present embodiment includes a light source unit 11, a lens 13, a slit-shaped member 15A, lenses 17A and 17B, a mirror 18, a perforated mirror 20, and an objective lens 22. The light receiving optical system 10B includes the objective lens 22, the perforated mirror 20, lenses 25A and 25B, a slit-shaped member 15B, and a light receiving element 28. The perforated mirror 20 is an optical path coupling portion that couples an optical path of the irradiation optical system 10A to the optical path of the light receiving optical system 10B. The perforated mirror 20 reflects the illumination light emitted from the light source unit 11 toward the objective lens 22 side (that is, subject eye E side), and allows a part of light, among the light (for example, fundus reflection light and the like from the subject eye E) from the objective lens 22, passing through an opening to pass toward the light receiving element 28 side. A beam splitter other than the perforated mirror 20 can also be used for the optical path coupling portion. For example, instead of the perforated mirror 20, a mirror in which a light-transmissive portion and a reflection portion are the reverse of the perforated mirror 20 may be used as the optical path coupling portion. In this case, an independent optical path of the light receiving optical system 10B is placed on a reflection side of a mirror, and an independent optical path of the irradiation optical system 10A is placed on a light-transmissive side of the mirror. Further, the perforated mirror 20 and a mirror used for alternative means of the perforated mirror 20 may be replaced with a combination of a half mirror and a light blocking portion.

In the present embodiment, the light source unit 11 includes a plurality of types of light sources having different wavelength bands. For example, the light source unit 11 includes visible light sources 11A and 11B, and infrared light sources 11C and 11D. The visible light sources 11A and 11B emit visible light, as the illumination light, including a plurality of wavelength regions. Thus, a color image of the fundus is captured by the illumination light emitted from the visible light sources 11A and 11B. The visible light sources 11A and 11B may be, for example, white light sources or light sources in which a plurality of monochromatic light sources, having different emission wavelengths, are combined. The light source unit 11 of the present embodiment includes two visible light sources and two infrared light sources. The two visible light sources 11A and 11B and the two infrared light sources 11C and 11D (hereinafter, may be simply referred to as “two light sources”) are arranged apart from an image capturing optical axis L on a pupil conjugate surface. Two light sources are arranged in parallel in the X direction that is a scan direction of FIG. 2 and are arranged in axial symmetry with respect to the image capturing optical axis L. As illustrated in FIG. 2, each of outer circumferential shapes of the two light sources may be a rectangular shape in which a direction crossing the scan direction is longer than the scan direction.

The slit-shaped member 15 is irradiated with light, which passes through the lens 13, from two light sources. In the present embodiment, the slit-shaped member 15A includes a light-transmissive portion (opening) formed in elongated shape in the Y direction. As a result, the illumination light is formed in a slit shape on the fundus conjugate surface (a region illuminated in the slit shape on the fundus is denoted by a symbol B). That is, the slit-shaped member 15A of the present embodiment functions as a slit formation portion that forms the illumination light in the slit shape on the fundus of the subject eye E.

The slit-shaped member 15A is displaced by the drive portion 15C such that the light-transmissive portion crosses the image capturing optical axis L in the X direction. Thereby, scan of the illumination light of the present example is achieved. In the present example, the scan is performed by the slit-shaped member 15B on the light receiving system side. In the present example, slit-shaped members on a light projection side and a light receiving side are driven in conjunction with each other by one drive portion (actuator) 15C. As described above, the slit-shaped members 15A and 15B and the drive portion 15C of the present embodiment function as a scan portion that scans slit-shaped illumination light in a direction (X direction) crossing (vertically crossing in the present embodiment) an extension direction (Y direction) of a slit.

In the irradiation optical system 10A, an image of each light source is relayed by an optical system from the lens 13 to the objective lens 22 to be formed on the pupil conjugate surface. That is, images of two light sources are formed at positions separated in a scan direction, on the pupil conjugate surface. By doing so, in the present embodiment, two light projection regions P1 and P2 on the pupil conjugate surface are formed as images of two light sources.

Further, slit-shaped light passing through the slit-shaped member 15A is relayed by an optical system from the lens 17A to the objective lens 22 to form an image on a fundus ER. Thereby, the illumination light is formed in the slit shape, on the fundus ER. The illumination light is reflected on the fundus ER and is extracted from a pupil EP.

Here, since an opening of the perforated mirror 20 conjugates with the pupil of the subject eye E, fundus reflection light used for capturing the fundus image is limited to a part of the fundus reflection light that passes through an image (pupil image) of the opening of the perforated mirror 20 on the pupil of the subject eye E. As such, an image of the opening on the pupil of the subject eye becomes a light receiving region R in the present example. The light receiving region R is a region, on the pupil, through which the fundus reflection light guided to the light receiving element 28 passes, among the fundus reflection light of the illumination light. That is, the perforated mirror 20 is an example of a light blocking member that allows reflection light from the light receiving region R to pass through the light blocking member toward an imaging surface side of the light receiving element 28 and blocks other light. The light receiving region R is formed to be interposed between two light projection regions P1 and P2 (images of two light sources). Further, the light receiving region R and the two light projection regions P1 and P2 are formed so as not to overlap each other on the pupil, as a result of appropriate setting of an image formation magnification of each image, a diameter of the opening, and arrangement interval between the two light sources. Specifically, in the present embodiment, the light receiving region R is formed between the two light projection regions P1 and P2. Thereby, occurrence of a white spot is reduced. Further, the two light projection regions P1 and P2 are formed symmetrically to the image capturing optical axis L.

The light (for example, the fundus reflection light and the like) passing through the objective lens 22 and the opening of the perforated mirror 20 forms an image of a slit-shaped region of the fundus ER at the fundus conjugate position via the lenses 25A and 25B. At this time, the light-transmissive portion of the slit-shaped member 15B is disposed at a position in which an image is formed, and thereby, harmful light is removed. That is, the slit-shaped member 15B is an example of a harmful light removing portion that removes light from a region other than a local effective region which is a part of an image capturing range.

The light receiving element 28 is disposed at the fundus conjugate position. In the present example, a relay system 27 is provided between the slit-shaped member 15B and the light receiving element 28. By the relay system 27, both the slit-shaped member 15B and the light receiving element 28 are arranged at the fundus conjugate position. As a result, both removal of the harmful light and image formation are satisfactorily performed. Instead of this, the relay system 27, which is provided between the light receiving element 28 and the slit-shaped member 15B, may be omitted, and both the light receiving element 28 and the slit-shaped member 15B may be arranged to be close to each other. In the present embodiment, a device having a two-dimensional light receiving surface is used as the light receiving element 28. For example, the light receiving element 28 may be a CMOS, a two-dimensional CCD, or the like. An image of the slit-shaped region of the fundus ER, which is formed on the light-transmissive portion of the slit-shaped member 15B, is projected to the light receiving element 28. The light receiving element 28 of the present embodiment has sensitivity in both infrared light and visible light.

In the present example, as the slit-shaped illumination light is scanned on the fundus ER, images (slit-shaped images) of scan positions on the fundus ER are sequentially projected for each scan line of the light receiving element 28. As such, an entire image of a scan range is projected onto the light receiving element 28 in a time-division manner. As a result, a two-dimensional front image of the fundus ER is captured as the entire image of the scan range.

Between the objective lens 22 and the subject eye E, the light receiving optical system 10B of the present embodiment guides the imaging light including reflection light of the illumination light from the fundus to the light receiving element 28, by passing through an optical path separated from an optical path of illumination light illuminated to the fundus (that is, not crossing and overlap an optical path of the illumination light). Accordingly, regardless of the diopter correction amount, a possibility that the reflection light, reflected by a cornea or the like of the subject eye E is guided to the light receiving element 28 is further reduced. Thus, a possibility that artifact occur in the fundus image is more appropriately suppressed.

The scan portion in the present embodiment is a device that mechanically scans the slit. However, a configuration of the scan portion can also be changed. For example, the scan portion on the light receiving optical system 10B side may be a device that electronically scans the slit. As an example, in a case where the light receiving element 28 is a CMOS, the slit may be scanned by a rolling shutter function of the CMOS. In this case, by displacing a region exposed on an imaging surface in synchronization with the scan portion in a light projection system, image capturing can be efficiently performed while removing the harmful light. Further, a liquid crystal shutter or the like can also be used as the scan portion that electronically scans the slit. Further, the scan portion in the irradiation optical system 10A may be an optical scanner (for example, a galvanometer mirror, an acoustic optical element, or the like) that changes a deflection direction of the illumination light. Further, an optical chopper, that scans the slit by rotating a wheel having a plurality of slits formed on an outer circumference, may be used as the scan portion. Further, the scan portion may be disposed on a common optical path of the irradiation optical system 10A and the light receiving optical system 10B.

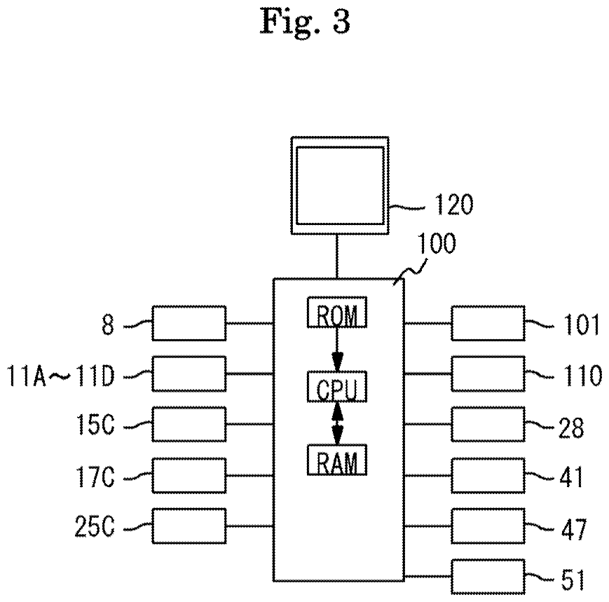

The image capturing optical system 10 includes a diopter correction portion. In the present example, diopter correction portions (diopter correction optical systems 17 and 25) are respectively provided on the independent optical path of the irradiation optical system 10A and the independent optical path of the light receiving optical system 10B. In the following, for the sake of convenience, a diopter correction optical system on an irradiation side (that is, performs diopter correction of the illumination light) is referred to as an irradiation side diopter correction optical system 17, and the diopter correction optical system on the light receiving side (that is, performs diopter correction of the imaging light) is referred to as a light receiving side diopter correction optical system 25. The irradiation side diopter correction optical system 17 of the present embodiment includes the lens 17A, the lens 17B, and a drive portion 17C (see FIG. 3). Further, the light receiving side diopter correction optical system 25 of the present example includes the lens 25A, the lens 25B, and a drive portion 25C (see FIG. 3). An interval between the lens 17A and the lens 17B is changed in the irradiation side diopter correction optical system 17, and an interval between the lens 25A and the lens 25B is changed in the light receiving side diopter correction optical system 25. As a result, diopter correction is performed in each of the irradiation optical system 10A and the light receiving optical system 10B. As described above, in the present embodiment, the drive portion 17C of the irradiation optical system 10A and the drive portion 25C of the light receiving optical system 10B can be driven independently. However, the diopter correction of the irradiation optical system 10A and the diopter correction of the light receiving optical system 10B may be performed in synchronization with each other.

In the present example, each of the irradiation side diopter correction optical system 17 and the light receiving side diopter correction optical system 25 includes an telecentric optical system. Each telecentric optical system maintains an image height in a region on an image side even when the diopter correction amount is changed. Thereby, a positional relation, on the fundus, between a slit opening of the irradiation optical system and a slit opening of the light receiving optical system can be kept constant regardless of a balance between an irradiation side diopter correction amount and a light receiving side diopter correction amount. Accordingly, the slit opening of the irradiation optical system and the slit opening of the light receiving optical system can be constantly matched on the fundus, regardless of the balance between the irradiation side diopter correction amount and the light receiving side diopter correction amount. Further, a change in an image size according to a change in the diopter correction amount can be suppressed.

The image capturing optical system 10 includes a split index projection optical system 50 as an example of a focus index projection optical system. The split index projection optical system 50 projects two split indexes onto the fundus. The split indexes are used for detecting a focus state. Further, in the present embodiment, the diopter correction amount (that is, refractive index of the subject eye E) is acquired from detection results of the focus state.

The split index projection optical system 50 may at least include, for example, a light source 51 (infrared light source), an index plate 52, and a declination prism 53. In the present embodiment, the index plate 52 is disposed at a position corresponding to an imaging surface of the light receiving element 28. Likewise, the index plate is also disposed at a position corresponding to each of the slit-shaped members 15A and 15B. In detail, in a case where the diopter correction amounts on the irradiation side and the light receiving side are 0 D, the index plate 52 is disposed at a position approximately conjugate with the fundus of an emmetropic eye (eye of 0 D). The declination prism 53 is disposed closer to the index plate 52 on the subject eye side than the index plate 52.

The index plate 52 forms, for example, slit light as an index. The declination prism 53 separates an index light flux via the index plate 52 to form a split index. The separated split index is projected onto the fundus of a subject eye via the irradiation side diopter correction optical system 17 to the objective lens 22. Accordingly, the split index is included in the fundus image (for example, fundus observation image).

In a case where the index plate 52 is not disposed in the fundus conjugate position, two split indexes on the fundus are separated, and in a case where the index plate 52 is disposed in the fundus conjugate position, the two split indexes match. A conjugate relation is adjusted by the irradiation side diopter correction optical system 17 disposed between the declination prism 53 and the subject eye E. Therefore, in the present example, defocusing is performed while matching the irradiation side diopter correction amount and the light receiving side diopter correction amount. At this time, a separated state of the split index indicates a focus state. By adjusting each of the diopter correction amounts on the irradiation side and the light receiving side such that the two split indexes match, each of the imaging surface and the slit-shaped members 15A and 15B has a conjugate positional relation with the fundus.

A refractive index of the subject eye E can be derived from the diopter correction amount when each of the imaging surface and the slit-shaped members 15A and 15B has a conjugate positional relation with the fundus. Therefore, in the present embodiment, an encoder (not illustrated) that reads at least one of an interval between the lens 17A and the lens 17B or an interval between the lens 25A and the lens 25B may be further included, and the refractive index of the subject eye E may be acquired based on a signal from the encoder.

The front eye portion observation optical system 40 shares the objective lens 22 and a dichroic mirror 43 with the image capturing optical system 10. The front eye portion observation optical system 40 further includes a light source 41, a half mirror 45, a light receiving element 47, and the like. The light receiving element 47 is a two-dimensional imaging element and is disposed at a position that optically conjugates with, for example, the pupil EP. The front eye portion observation optical system 40 illuminates a front eye portion with infrared light emitted from the light source 41 and captures a front image of the front eye portion. The front eye portion observation optical system 40 illustrated in FIG. 2 is only an example and may capture the front eye portion on an optical path independent of other optical systems.

### Control System

A control system of the fundus capturing apparatus (ocular fundus image processing apparatus) 1 will be described with reference to FIG. 3. In the present embodiment, a controller 100 controls respective portions of the fundus capturing apparatus 1. Further, as described above, it is assumed that image processing of various images obtained by the fundus capturing apparatus 1 is also executed by the controller 100. In other words, in the present embodiment, the controller 100 also serves as an image processing portion.

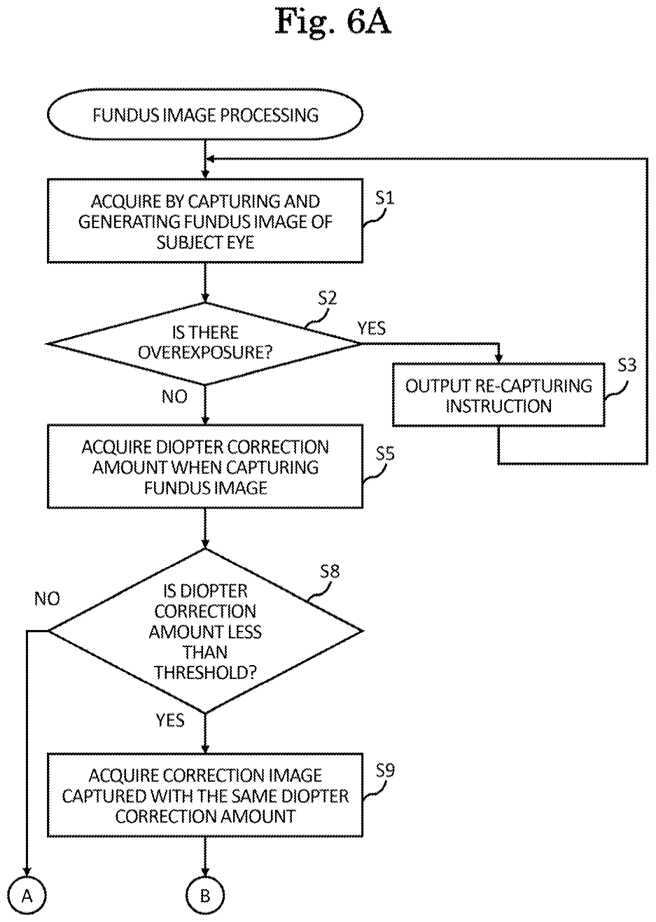

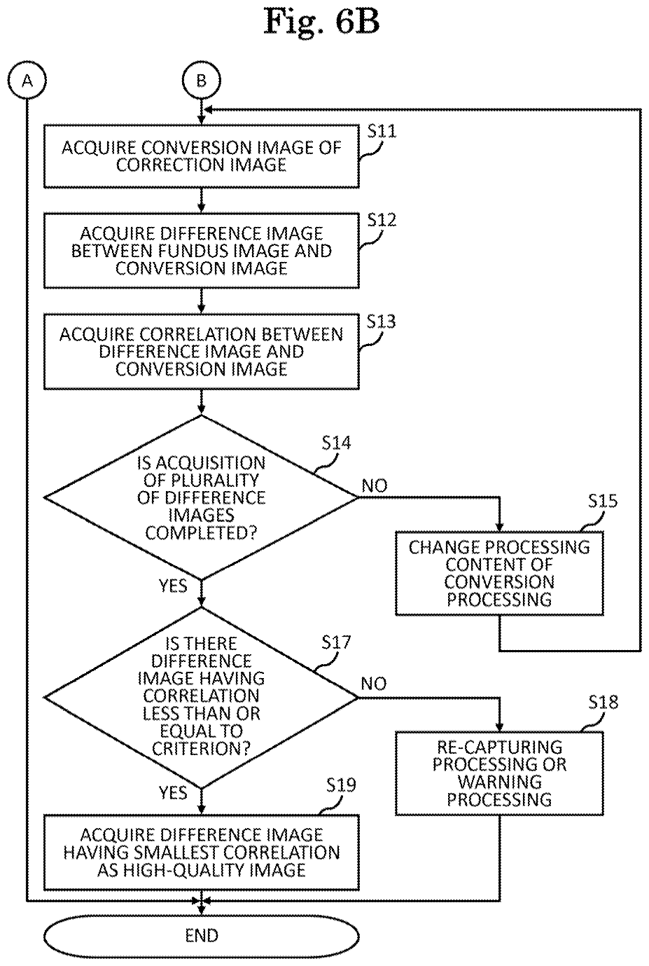

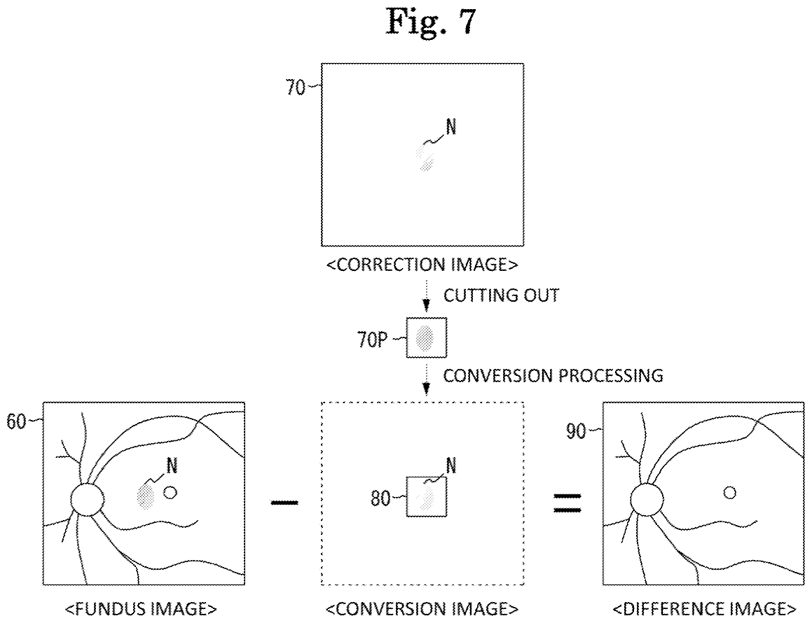

The controller 100 is a processing apparatus (processor) including an electronic circuit that executes control processing of each portion and arithmetic processing. The controller 100 is implemented by a central processing unit (CPU), a memory, and the like. The controller 100 is electrically connected to a storage portion 101 through a bus or the like. The storage portion (storage apparatus) 101 stores various control programs, fixed data, and the like. In the present embodiment, a fundus image processing program for executing fundus image processing (see FIGS. 6A and 6B) and data of a plurality of correction images 70 (see FIG. 7) (hereinafter, may be simply referred to as a “correction image”) are stored in the storage portion 101. Further, the storage portion 101 may store temporary data or the like. An image captured by the fundus capturing apparatus 1 may be stored in the storage portion 101. However, the present disclosure is not limited thereto, and the captured image may be stored in an external storage apparatus (for example, a storage apparatus connected to the controller 100 through a LAN and a WAN).

The controller 100 is also electrically connected to the respective portions such as the drive portion 8, the light sources 11A to 11D, the drive portion 15C, the drive portion 17C, the drive portion 25C, the light receiving element 28, the light source 41, the light receiving element 47, the light source 51, an input interface 110, and the monitor 120. Further, the controller 100 controls the respective portions described above based on an operation signal output from the input interface 110. The input interface 110 is an example of an operation input portion that receives an operation of an examiner. The input interface 110 may be, for example, a mouse, a keyboard, or the like.

### Artifact

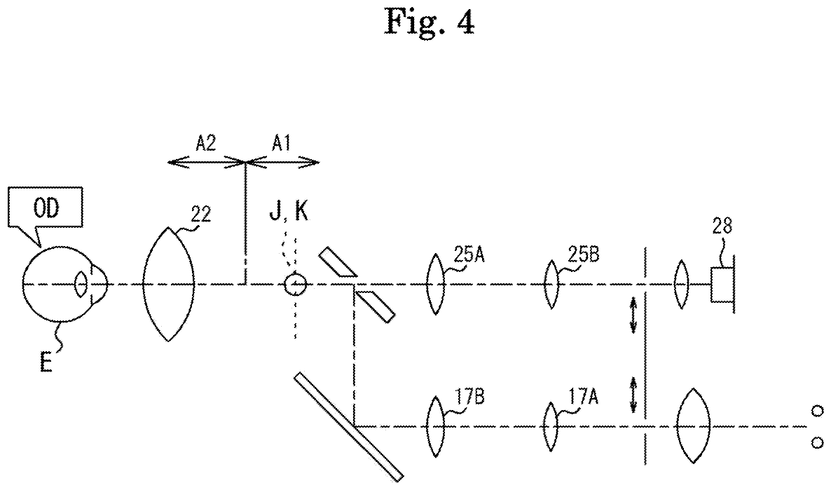

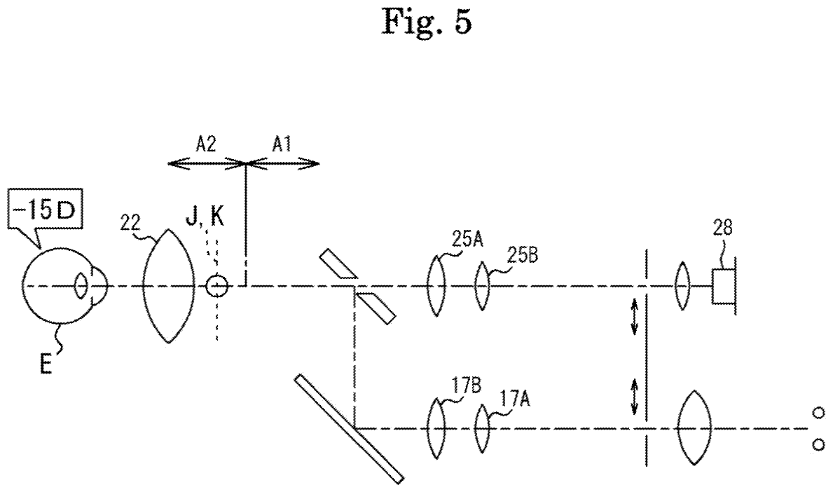

A cause of artifact in a fundus image 60 (see FIG. 7) of the present embodiment will be described with reference to FIGS. 4 and 5. As illustrated in FIGS. 4 and 5, a region where a distance from the objective lens 22 to an upstream side of an optical path of the illumination light is greater than or equal to a threshold is assumed as a region A1. A region where the distance from the objective lens 22 to the upstream side of the optical path of the illumination light is less than the threshold is assumed as a region A2. In the fundus capturing apparatus 1 of the present embodiment, in a case where a position conjugate with the fundus with respect to the objective lens 22 (that is, a position J of an intermediate image surface of the fundus) and a light collection position K of the illumination light are located in the region A1 the amount of the illumination light reflected by the objective lens 22 and guided to the light receiving element 28 is hard to increase. Meanwhile, when the position J of the intermediate image surface of the fundus and the light collection position K of the illumination light are located in the region A2, the reflection light of the illumination light from the objective lens 22 is easily guided to the light receiving element 28. As a result, as a result, as illustrated in FIG. 7, artifact (white spot) N due to the reflection light of the illumination light from the objective lens 22 is likely to occur in the fundus image 60 to be captured. The more the refractive index of the subject eye E is a value on a minus diopter side, the stronger the influence of artifact N.

As illustrated in FIG. 4, in a case where the subject eye E is an emmetropic eye (that is, when the refractive index of the subject eye E is 0 D (diopter)), the position J of the intermediate image surface of the fundus and the light collection position K of the illumination light is located in the region A1. Thus, the artifact is unlikely to occur in the fundus image 60.

However, as illustrated in FIG. 5, in a case where a subject eye is a myopic eye (in the example illustrated in FIG. 5, in a case where the refractive index of the subject eye E is −15 D), when the diopter correction is performed by the diopter correction portion (in the present embodiment, the lenses 17A and 17B, the lenses 25A and 25B, and the like), the position J of the intermediate image surface of the fundus and the light collection position K of the illumination light are located in the region A2. As a result, the artifact N caused by the reflection light of the illumination light from the objective lens 22 is likely to occur in the fundus image 60.

In the present embodiment, the artifact N caused by the reflection light of the illumination light from the objective lens 22 is exemplified to describe the artifact in the fundus image 60. However, the artifact can also be caused by various types of reflection and scattered light generated in a common optical path of the illumination light and the imaging light. According to the technique exemplified in the present disclosure, it is possible to reduce an influence of not only the artifact caused by the reflection light of the illumination light from the objective lens 22 but also the artifact caused by various types of light generated in the common optical path of the illumination light and the imaging light.

### Fundus Image Processing

Fundus image processing of the present embodiment will be described with reference to FIGS. 6A, 6B, and 7. The fundus image processing exemplified in FIGS. 6A and 6B is executed by the controller 100 of the fundus capturing apparatus (ocular fundus image processing apparatus) 1 according to a fundus image processing program stored in the storage portion 101.

As illustrated in FIG. 6A, the controller 100 acquires the fundus image 60 of a subject eye (S1). In the present embodiment, the controller of the fundus capturing apparatus 1 performs an operation of capturing the fundus image 60 of the subject eye and generates data of the fundus image 60 by executing an imaging process for a light receiving signal output when the light receiving element 28 (see FIG. 2) receives light (imaging light).

The controller 100 determines whether or not there is a saturation (overexposure) in the artifact N (see FIG. 7) in the fundus image 60 acquired in S1 (S2). In the present embodiment, in a case where a luminance value of the artifact N in the fundus image 60 is greater than or equal to a threshold, the controller 100 determines that there is the saturation (overexposure) in the artifact N. In a case where there is the saturation (overexposure) in the artifact N (S2: YES), even when image processing using the correction image 70 is executed, it is difficult to restore an image of a fundus tissue in a part where the artifact N and the fundus image overlap each other. Thus, the controller 100 outputs an instruction for prompting a user to re-capture a fundus of the subject eye (S3), and the processing returns to S1. For example, the re-capturing instruction may be output by displaying a message on the monitor 120 or may be output as voice.

When there is no saturation (overexposure) in the fundus image 60 (S2: NO), the controller 100 acquires the diopter correction amount (that is, focus adjustment amount) when the fundus image 60 is captured (S5). As described above, in the present embodiment, the diopter correction amount is acquired based on detection results of the encoder provided in the diopter correction portion (diopter correction optical systems 17 or 25). The diopter correction may be performed in conjunction with each of the illumination light and the imaging light or may be performed separately from each of the illumination light and the imaging light.

The controller 100 determines whether or not the diopter correction amount (at least the diopter correction amount of the illumination light in the present embodiment) is less than a threshold (that is, a value on a minus diopter side rather than the threshold) when the fundus image 60 is captured (S8). As described above, in the fundus capturing apparatus 1 of the present embodiment, the more the refractive index (that is, diopter correction amount corrected by the diopter correction portion) of the subject eye E is the value on the minus diopter side, the stronger the influence of the artifact N. Meanwhile, when the diopter correction amount is the value on the plus diopter side, the artifact N is unlikely to occur. Thus, in a case where the diopter correction amount is not a value on the minus diopter side of the threshold (S8: NO), the controller 100 does not execute the processing (S9 to S19) for suppressing the influence of the artifact N of the fundus image 60. The threshold referred to in S8 may be appropriately set according to the diopter correction amount and a degree of the artifact N. In the present embodiment, as illustrated in FIGS. 4 and 5, the diopter correction amount becomes the threshold referred to in S8, when the light collection position K of the illumination light reaches a boundary between the region A1 where the artifact N is unlikely to occur and the region A2 where the artifact N occurs.

In a case where the diopter correction amount is larger than the threshold (S8: YES), the controller 100 acquires the correction image 70 (see FIG. 7) captured with the same diopter correction amount as the diopter correction amount acquired in S5 (S9). The correction image 70 is an image captured by emitting the illumination light in a state where light from a downstream side of the objective lens 22 (see FIG. 2) in an optical path of the illumination light toward the objective lens 22 is blocked. That is, the correction image 70 is an image that includes only artifact caused by the illumination light without including the fundus of the subject eye as a capturing target and. A method of capturing the correction image 70 by the fundus capturing apparatus 1 can be appropriately selected. For example, the correction image 70 may be captured by capturing an image in a state where the downstream side of the illumination light rather than the objective lens 22 is blocked by a cover. Further, the correction image 70 may be captured by capturing a blackout curtain or the like. The diopter correction amount acquired in S5 and the diopter correction amount at the time of capturing the correction image 70 may not be completely the same as each other, and a difference between both may be less than or equal to the threshold.

As illustrated in FIG. 7, the correction image 70 includes the artifact N. The artifact N included in the image changes according to the diopter correction amount (that is, focus adjustment amount). Thus, the artifact N of the correction image 70 captured with the diopter correction amount acquired in S5 and the artifact N included in the fundus image 60 acquired in S1 are similar to each other.

In the present embodiment, a plurality of correction images 70 captured by the fundus capturing apparatus 1 while changing the diopter correction amount are previously stored in the storage portion 101. In S9, the controller 100 acquires the correction image 70 captured by the diopter correction amount in which a difference from the diopter correction amount acquired in S5 is less than or equal to (for example, same) a threshold, among the plurality of correction images 70 stored in the storage portion 101. Thus, there is no need to capture the correction image 70 each time the fundus image 60 is captured. However, the correction image 70 may be acquired by also capturing the correction image 70 when the fundus image 60 is captured.

Next, the controller 100 acquires the conversion image 80 (see FIG. 7) by executing conversion processing of converting a pixel value of the correction image 70 acquired in S9 (S11). A method of the conversion processing can be appropriately selected. As an example, in the present embodiment, the conversion image 80 is acquired by executing gamma correction on each pixel value of the correction image 70 by using a lookup table. However, processing other than the gamma correction (for example, at least one of brightness correction, contrast enhancement, histogram extension, histogram equalization, and the like) may be executed as the conversion processing. Further, in S11 of the present embodiment, different processing is executed for each channel (for example, RGB channel in the present embodiment) of the pixel of the correction image 70. Thus, many types of the conversion processing can be executed on the correction image 70. However, the same processing may be executed on each of the channels.

Further, as illustrated in FIG. 7, in S11, the controller 100 of the present embodiment acquires a partial correction image 70P obtained by cutting out a partial region, from the entire image region of the correction image 70, including the artifact N. The controller 100 acquires the conversion image 80 by executing conversion processing for the partial correction image 70P. Thus, since the conversion processing for a region where the artifact

N does not exist is omitted, the amount of processing is appropriately reduced.

Next, in a state where the fundus image 60 acquired in S1 and the conversion image 80 (conversion image 80 of the partial correction image 70P in the present embodiment) acquired in S11 are aligned, the controller 100 acquires the difference image 90 by taking a difference between respective pixels (S12). The artifact N of the fundus image 60 and the artifact N of the correction image 70 are similar to each other. Thus, when conversion from the correction image 70 into the conversion image 80 is properly executed, the difference image 90 becomes an image in which the artifact N is removed from the fundus image 60.

Next, the controller 100 acquires a correlation between the difference image 90 acquired in S12 and the conversion image 80 (conversion image 80 of the partial correction image 70P in the present embodiment) acquired in S11 (S13). In a case where the influence of the artifact N remains in the difference image 90, the correlation between the difference image 90 and the conversion image 80 increases. Meanwhile, when the influence of the artifact N remaining in the difference image 90 is small, the correlation between the difference image 90 and the conversion image 80 is reduced. Thus, by using the correlation between the difference image 90 and the conversion image 80, it is appropriately determined whether or not the influence of the artifact N in the fundus image 60 is suppressed. The controller 100 may acquire the correlation between the difference image 90 and the correction image 70. When the influence of the artifact N remaining in the difference image 90 is small, the correlation between a difference image 90 and the correction image 70 is reduced. Thus, even in this case, it is appropriately determined whether or not the influence of the artifact N in the fundus image 60 is suppressed.

In the present embodiment, a correlation in a region of the conversion image 80 is acquired in a state where the difference image 90 and the conversion image 80, based on the partial correction image 70P, are aligned. Thus, the amount of processing is appropriately reduced as compared with a case where a correlation of the entire image region is acquired.

Next, the controller 100 determines whether or not to end the acquisition processing of the plurality of difference images 90 (S14). For example, in a case where it is determined that the difference image 90 having the smallest correlation between the difference image 90 and the correction image 70 is previously acquired, the controller 100 may end the acquisition processing of the plurality of difference images 90. Further, the controller 100 may end the acquisition processing of the difference images 90 when a predetermined number of the difference images 90 are acquired. In a case where the acquisition processing of the difference image 90 is not completed (S14: NO), the controller 100 changes processing content of the conversion processing executed in S11 (S15) and executes the processing of S11 to S13 again.

When the acquisition processing of the plurality of difference images 90 is completed (S14: YES), the controller 100 determines whether or not there is the difference image 90 of which a correlation with the conversion image 80 is less than or equal to a criterion, among the acquired plurality of difference images 90 (S17). That is, the controller 100 searches for the difference image 90 whose correlation with the conversion image 80 is less than or equal to the criterion, among the plurality of difference images 90. The criterion referred to in S17 may be appropriately set according to a degree that the influence of the artifact N is suppressed in the difference image 90 and the correlation between the difference image 90 and the conversion image 80. In a case where there is no difference image 90 whose correlation is less than or equal to the criterion (S17: NO), there is a possibility that at least one of the fundus image 60 and the correction image 70 is not appropriately captured. Thus, the controller 100 executes re-capturing processing or a warning processing (S18). In the re-capturing processing, at least one of the fundus image 60 and the correction image 70 is re-captured, and the processing returns to S2. In the warning processing, a user is warned that image quality of the fundus image (in this case, the difference image 90 in which the influence of the artifact N is suppressed) is low. For example, the warning may be output by displaying a message on the monitor 120 or may be output as voice.

If there is the difference image 90 whose correlation is less than or equal to the criterion, among the plurality of acquired difference images 90 (S17: YES), at least one of the difference images 90 whose correlation is less than or equal to the criterion is acquired as a high-quality image in which the influence of the artifact N is suppressed (S19). Specifically, in S19 of the present embodiment, the difference image 90 having the smallest correlation with the conversion image 80 is acquired as the high-quality image, among the plurality of acquired difference images 90. The controller 100 may acquire the difference image 90, as the high-quality image, whose correlation with the correction image 70 is less than or equal to the criterion.

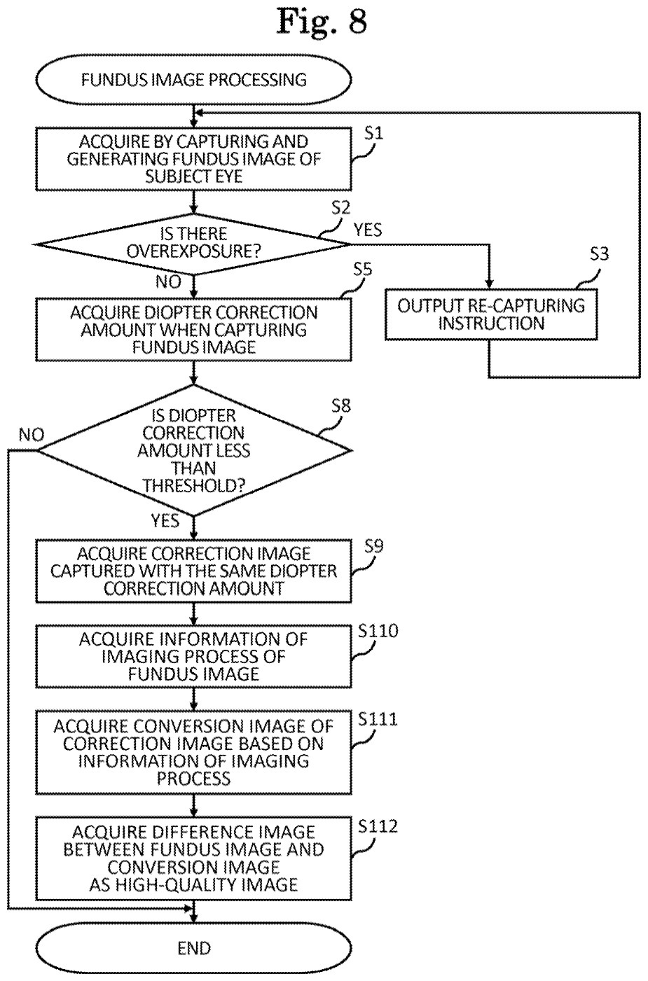

A modification example of the fundus image processing exemplified in FIGS. 6A and 6B will be described with reference to FIG. 8. As described above, various types of processing are often executed in an imaging process of generating image data based on the light receiving signal output by the light receiving element 28 of the fundus capturing apparatus 1. In the modification example illustrated in FIG. 8, the correction image 70 is converted into the conversion image 80 based on information of the imaging process, and the difference between the fundus image 60 and the conversion image 80 is taken, and thereby, the high-quality image is acquired. For some steps (S1, S2, S3, S5, S8, and S9) of the modification example illustrated in FIG. 8, the same processing as each step of the fundus image processing described above (see FIG. 6A) can be adopted. Thus, among the plurality of steps illustrated in FIG. 8, steps that can adopt the same processing as the steps illustrated in FIGS. 6A and 6B are denoted by the same step numbers as in FIGS. 6A and 6B, and description thereof will not be repeated or will be simplified.

As illustrated in FIG. 8, in the fundus image processing of the modification example, the controller 100 acquires the correction image 70 (see FIG. 7) captured with the diopter correction amount in which a difference from the diopter correction amount acquired in S5 is less than or equal to (for example, same) a threshold (S9). The controller 100 acquires the information of the imaging process (for example, at least one of gamma correction, gain adjustment, and the like) when the data of the fundus image 60 is generated based on the light receiving signal output by the light receiving element 28 (see FIG. 2) (S110).

Next, the controller 100 acquires the conversion image 80 by executing the conversion processing of converting a pixel value of the correction image 70 based on the information of the imaging process acquired in S110 (S111). That is, in S111, the correction image 70 is converted based on the information of the imaging process when generating the fundus image 60 such that there is no difference between the imaging process at the time of generating the fundus image 60 and the imaging process at the time of generating the correction image 70. As a result, the artifact N included in the fundus image 60 is similar to the artifact N included in the conversion image 80. The controller 100 acquires the difference image 90 of the fundus image 60 acquired in S1 and the conversion image 80 acquired in S111, as a high-quality image in which the influence of the artifact N of the fundus image 60 is suppressed (S112).

The techniques disclosed in the embodiments described above are merely examples. Thus, the techniques exemplified in the embodiments can also be modified. For example, some of the plurality of types of processing exemplified in FIGS. 6A, 6B and 8 can also be omitted. Specifically, the processing of S2 and S3 in FIGS. 6A and 8 may be omitted. The processing of S8 in FIGS. 6A and 8 may be omitted.

The processing of acquiring the fundus image 60 in S1 of FIGS. 6A and 8 is an example of “fundus image acquisition processing”. The processing of acquiring the correction image 70 in S9 of FIGS. 6A and 8 is an example of “correction image acquisition processing”. The processing of acquiring the conversion image in S11 of FIG. 6B and S111 of FIG. 8 is an example of “conversion image acquisition processing”. In S12 to S15 and S19 of FIG. 6B, the processing of acquiring the difference image 90, as a high-quality image, in which an influence of the artifact is less than or equal to a criterion is an example of “high-quality image acquisition processing”. The processing of acquiring the diopter correction amount in S5 of FIGS. 6A and 8 is an example of “diopter correction amount acquisition processing”. The processing of acquiring the information of the imaging process in S110 of FIG. 8 is an example of “imaging information acquisition processing”. In S111 of FIG. 8, the processing of acquiring the conversion image 80 based on the information of the imaging process is an example of “conversion image acquisition processing”. The processing of acquiring the difference image 90 as a high-quality image in S112 of FIG. 8 is an example of “high-quality image acquisition processing”.

What is claimed is:

1. An ocular fundus image processing apparatus that processes a fundus image captured by a fundus capturing apparatus,
wherein the fundus capturing apparatus comprises:
a light source that emits illumination light;
an objective lens that irradiates a downstream side of an optical path with the illumination light; and
a light receiving element that receives imaging light incident from the objective lens, and
the ocular fundus image processing apparatus comprises a controller configured to execute:
fundus image acquisition processing of acquiring a fundus image, which is an image of a fundus of a subject eye, captured by the fundus capturing apparatus;
correction image acquisition processing of acquiring a correction image, which is an image including artifact caused by the illumination light, captured by the fundus capturing apparatus without including the fundus of the subject eye as a capturing target;
conversion image acquisition processing of executing conversion processing of converting a pixel value of the correction image a plurality of times while changing processing content to acquire a plurality of conversion images; and
high-quality image acquisition processing of acquiring a difference image, as a high-quality image, in which an influence of the artifact is suppressed to be less than or equal to a criterion, among a plurality of difference images acquired by taking a difference between each of the plurality of conversion images and the fundus image.

2. The ocular fundus image processing apparatus according to claim 1,
wherein, in the high-quality image acquisition processing, the controller acquires a difference image, as the high-quality image, of which a correlation with the conversion image or the correction image is less than or equal to a criterion, among the plurality of difference images.

3. The ocular fundus image processing apparatus according to claim 1,
wherein the fundus capturing apparatus further comprises a diopter correction portion that performs diopter correction according to the subject eye in conjunction with each of the illumination light and the imaging light or separately from each of the illumination light and the imaging light, and
the correction image, acquired in the correction image acquisition processing, is an image captured in a state where diopter correction is made with a diopter correction amount in which a difference from a diopter correction amount obtained by the diopter correction portion when the fundus image is captured is less than or equal to a threshold.

4. The ocular fundus image processing apparatus according to claim 3,
wherein the controller further executes:
diopter correction amount acquisition processing of acquiring the diopter correction amount by the diopter correction portion when the fundus image is captured; and
the controller executes the correction image acquisition processing, the conversion image acquisition processing, and the high-quality image acquisition processing only in a case where the diopter correction amount, acquired in the diopter correction amount acquisition processing, is a value on a minus diopter side from a threshold.

5. The ocular fundus image processing apparatus according to claim 3,
wherein a plurality of the correction images captured by the fundus capturing apparatus while changing the diopter correction amount by the diopter correction portion are previously stored in a storage apparatus, and
in the correction image acquisition processing, among the plurality of correction images stored in the storage apparatus, the correction image, which is captured in the state where the diopter correction is made with the diopter correction amount in which the difference from the diopter correction amount obtained by the diopter correction portion when the fundus image is captured is less than or equal to the threshold, is acquired.

6. The ocular fundus image processing apparatus according to claim 1,
wherein, in the high-quality image acquisition processing, in a case where the difference image in which an influence of the artifact is less than or equal to the criterion does not exist, the controller executes re-capturing processing of outputting a re-capturing instruction of at least one of the fundus image and the correction image, or warning processing of warning a user that image quality of the fundus image is low.

7. The ocular fundus image processing apparatus according to claim 1,
wherein the conversion processing, executed in the conversion image acquisition processing, includes at least one of gamma correction, brightness correction, contrast enhancement, histogram extension, and histogram equalization.

8. The ocular fundus image processing apparatus according to claim 1,
wherein the controller executes the conversion processing in the conversion image acquisition processing and processing of taking a difference between the fundus image and the conversion image in the high-quality image acquisition processing, for a partial region including artifact, in an entire image region of the fundus image and the correction image.
